(19) United States
(12) Patent Application Publication
(10) Pub. No.: US 2025/0375379 A1
(43) Pub. Date: Dec. 11, 2025

(54) TOPICAL POWDER VEHICLE FOR THE TREATMENT OF ALOPECIA

(71) Applicant: Yasmin Qaseem, Long Beach, CA (US)

(72) Inventor: Yasmin Qaseem, Long Beach, CA (US)

(21) Appl. No.: 19/226,433

(22) Filed: Jun. 3, 2025

Related U.S. Application Data
(60) Provisional application No. 63/657,239, filed on Jun. 7, 2024.

(51) Int. Cl. A61K 9/14; A61K 31/506

(52) U.S. Cl. CPC A61K 9/148; A61K 9/143; A61K 9/145; A61K 9/146; A61K 31/506

(57) ABSTRACT

A powder vehicle composition for topical minoxidil in which case the formulation is a powder, combining minoxidil with a dry shampoo to assist in the growth of hair on the scalp as well as absorb excess grease and oil from the scalp. The powder vehicle of minoxidil compounded with dry shampoo serves to provide similar hair growth benefits of topical minoxidil solution or foam while also removing excess grease and oil from the scalp. The instant invention contains as ingredients: minoxidil, an alcohol, arrowroot powder, cornstarch, starch, fragrance, silica, a paraffinic hydrocarbon, an alkane, oat flour, clay, and petroleum. The powder vehicle composition provides a viable solution for people with hair styling practices that do not permit the hair to become wet with liquid or foam on a daily basis.

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### CROSS-REFERENCE TO RELATED APPLICATIONS

This application is a Utility Patent application claiming priority to U.S. Provisional Patent Application Ser. No. 63/657,239, filed on Jun. 7, 2024, which is incorporated by reference herein in its entirety.

### BACKGROUND OF THE INVENTION

### 1) Field of the Invention

The invention relates to the field of dermatology, and more specifically to a delivery formulation for the treatment of alopecia.

### 2) Description of Related Art

Minoxidil is a medication that increases new hair growth in cases of hereditary hair loss or alopecia. Application is a topical solution or foam applied to the scalp to promote hair growth. A brand name of this medication is Rogaine®, common concentrations commercially vary from 2 to 15%.

Dry shampoo cleans hair by absorbing oil, sweat, and odor while also making hair smell clean with a crisp, time-released fragrance. Commercial dry shampoos usually come in a spray bottle. They are made with a base of alcohol or starch. When you spray the product into your hair, the alcohol or starch soaks up oil and grease, making it look cleaner.

Patients with alopecia from multiple causes may benefit from the use of topical minoxidil. However, existing formulations typically include liquid solutions or foams which may be difficult to incorporate in all hair styling practices. Moreover, this can lead to a buildup or residue if the scalp is not washed on a daily basis. Many people with alopecia prefer to wash their hair only a few times per week or month and may find this inconvenient. Additionally, people with hair styling practices that do not permit the hair to become wet with liquid or foam currently do not have an option to use the current formulations.

There remains a need in the art for a solution to at least one of the aforementioned problems with a combined solution.

### SUMMARY OF THE INVENTION

The present invention relates to topical administration carrier compositions that are usefully employed in preparing formulations of active ingredients for topical administration to the body, e.g., to the skin, hair, etc. Combination of a dry shampoo with the therapeutic benefits of minoxidil provides a solution address timing when a desired shampoo is not on a daily basis. Additionally, styling considerations where it may be preferred to inhibit daily shampooing is also addressed.

The instant invention in one form is directed to an application modality for topical minoxidil in which case the formulation is a powder, combining minoxidil powder with a dry shampoo to assist in the growth of hair on the scalp as well as absorb excess grease and oil from the scalp.

A powder vehicle composition of minoxidil compounded with dry shampoo serves to provide similar hair growth benefits of topical minoxidil solution or foam while also removing excess grease and oil from the scalp, allowing more convenient styling of hair.

The powder vehicle composition of the invention contains as ingredients minoxidil, an alcohol, arrowroot powder, cornstarch, starch, fragrance, silica, a paraffinic hydrocarbon, an alkane, oat flour, clay, and petroleum.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a flowchart showing the steps in producing a Minoxidil based dry shampoo.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms topical vehicle and carrier are used interchangeably to mean a formulation to the instant invention.

The instant invention in one form is directed to an application modality for topical minoxidil in which case the formulation is a powder, combining minoxidil powder with a dry shampoo to assist in the growth of hair on the scalp as well as absorb excess grease and oil from the scalp.

A powder vehicle composition of minoxidil compounded with dry shampoo serves to provide similar hair growth benefits of topical minoxidil solution or foam while also removing excess grease and oil from the scalp, allowing more convenient styling of hair.

The powder vehicle composition of the invention contains as ingredients minoxidil, an alcohol, arrowroot powder, cornstarch, starch, fragrance, silica, a paraffinic hydrocarbon, an alkane, oat flour, clay, and petroleum.

The powder vehicle composition provides a viable solution for people with hair styling practices that do not permit the hair to become wet with liquid or foam.

In another embodiment, the paraffinic hydrocarbon is butane.

In another embodiment, the alkane is isobutene.

In the instant invention the powder vehicle is distributed via an aerosol spray can.

In another embodiment, the powder vehicle is distributed via a pump spray bottle.

In another embodiment, the powder vehicle is distributed via a brush applicator.

Specific formulations of the powder vehicle is based on the method of distribution.

In some aspects, the present disclosure relates to a powder vehicle for the treatment of alopecia. The powder vehicle may include a variety of components, each serving a specific function in the overall formulation. In some cases, the powder vehicle may include minoxidil, a medication known for its ability to stimulate hair growth. The powder vehicle may also include an alcohol, which can serve as a solvent or carrier for other ingredients.

The powder vehicle may further include arrowroot powder, cornstarch, and starch. These components may serve as absorbents, helping to soak up excess oil and grease from the scalp. This can be particularly beneficial for individuals who prefer not to wash their hair daily, as it can help to keep the hair looking clean and fresh.

In addition to the aforementioned components, the powder vehicle may also include a fragrance. This can help to mask any unpleasant odors and provide a pleasant scent to the hair and scalp. The powder vehicle may also include silica, a component known for its ability to absorb moisture and oil.

The powder vehicle may include a paraffinic hydrocarbon and an alkane. These components may serve as propellants, helping to distribute the powder vehicle evenly across the scalp. The powder vehicle may also include oat flour, clay, and petroleum. These components may provide additional benefits, such as soothing the scalp, absorbing excess oil, and providing a protective barrier on the scalp, respectively. The powder vehicle may be formulated to provide a convenient and effective solution for the treatment of alopecia, offering the benefits of minoxidil in a dry, easy-to-use format that can be incorporated into a variety of hair care routines.

In some embodiments, the paraffinic hydrocarbon included in the powder vehicle may be butane. Butane, in this context, may serve as a propellant, aiding in the distribution of the powder vehicle when applied. In other embodiments, the alkane included in the powder vehicle may be isobutene. Similar to butane, isobutene may also function as a propellant, assisting in the even application of the powder vehicle across the scalp.

The alcohol component of the powder vehicle may be ethanol. Ethanol may serve multiple functions within the powder vehicle, including acting as a solvent for other ingredients, enhancing the penetration of minoxidil into the scalp, and aiding in the evaporation of the vehicle after application, among others.

The powder vehicle may include arrowroot powder, cornstarch, and starch in a ratio of 1:1:1. These components, in these proportions, may effectively absorb excess oil and grease from the scalp, contributing to the overall cleanliness and freshness of the hair.

The powder vehicle may include oat flour, clay, and petroleum in a ratio of 2:1:1. Oat flour may provide soothing properties to the scalp, clay may further assist in oil absorption, and petroleum may provide a protective barrier on the scalp, helping to lock in moisture and protect the scalp from environmental factors. The specific ratio of these components may be optimized to provide these benefits while maintaining the overall consistency and effectiveness of the powder vehicle.

The paraffinic hydrocarbon included in the powder vehicle may be specified as butane. Butane, in this context, may serve as a propellant, aiding in the distribution of the powder vehicle when applied. This can be particularly beneficial in embodiments where the powder vehicle is distributed via an aerosol spray can, as butane can help to ensure a fine, even spray of the powder vehicle across the scalp.

The alkane included in the powder vehicle may be specified as isobutene. Similar to butane, isobutene may also function as a propellant, assisting in the even application of the powder vehicle across the scalp. This can be particularly beneficial in embodiments where the powder vehicle is distributed via a pump spray bottle or a brush applicator, as isobutene can help to ensure a smooth, even distribution of the powder vehicle.

In some cases, the specific choice of paraffinic hydrocarbon and alkane, such as butane and isobutene, may be based on factors such as the desired pressure within the distribution container, the desired spray pattern, and the desired rate of evaporation of the powder vehicle after application, among others.

The powder vehicle may be applied to the scalp to assist in the growth of hair and absorb excess grease and oil from the scalp. This application may be particularly beneficial for individuals suffering from alopecia, as it can provide the hair growth benefits of minoxidil in a convenient, easy-to-use format. The powder vehicle may be applied directly to the scalp, where it can interact with the hair follicles to stimulate hair growth and absorb excess oil and grease, helping to keep the hair looking clean and fresh. In some cases, the powder vehicle may be applied to the scalp once a day. This frequency of application may be sufficient to provide the desired hair growth benefits, while also helping to maintain the cleanliness and freshness of the hair. However, the frequency of application may be adjusted based on individual hair care routines and personal preferences. In other cases, the powder vehicle may be applied to the scalp more than once a day. This increased frequency of application may provide enhanced hair growth benefits and increased oil and grease absorption. However, the specific frequency of application may be determined based on factors such as the severity of the alopecia, the individual's hair care routine, and the individual's personal preferences.

The powder vehicle may be applied to the scalp less frequently, such as every other day or a few times per week. This reduced frequency of application may still provide the desired hair growth benefits and oil and grease absorption, while also accommodating individuals who prefer to wash their hair less frequently. The specific frequency of application in these cases may be determined based on factors such as the individual's hair care routine, the individual's personal preferences, and the individual's response to the treatment.

The powder vehicle may be distributed via various means. For instance, in some cases, the powder vehicle may be distributed via an aerosol spray can. The aerosol spray can may be designed to release the powder vehicle in a fine mist, ensuring even coverage across the scalp. The use of an aerosol spray can may be particularly beneficial for individuals with longer or thicker hair, as it can help to ensure that the powder vehicle reaches the scalp effectively.

In other embodiments, the powder vehicle may be distributed via a pump spray bottle. The pump spray bottle may release the powder vehicle in a more concentrated spray, which may be beneficial for targeted application to specific areas of the scalp. The pump spray bottle may also offer more control over the amount of powder vehicle released with each spray, allowing for more precise application.

In yet other embodiments, the powder vehicle may be distributed via a brush applicator. The brush applicator may be designed to release the powder vehicle in a controlled manner, allowing for precise application to specific areas of the scalp. The brush applicator may also help to distribute the powder vehicle evenly across the scalp, ensuring that all areas of the scalp receive an adequate amount of the powder vehicle. The choice of distribution method may depend on various factors, such as the individual's hair type, the severity of the alopecia, the individual's personal preferences, and the individual's hair care routine. For instance, individuals with shorter hair or less severe alopecia may prefer the precision of a brush applicator, while individuals with longer hair or more severe alopecia may prefer the wide coverage of an aerosol spray can or pump spray bottle.

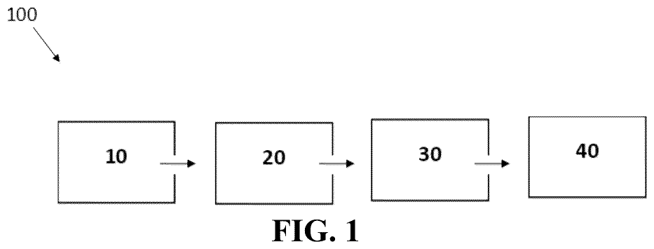

Referring now to the drawing, FIG. 1 is a flowchart 100 showing the steps to produce the minoxidil based dry shampoo portion 40. A minoxidil power portion 10 is mixed with a vehicle portion 20. The vehicle portion 20 includes ingredients for a dry shampoo, including an alcohol, arrowroot powder, cornstarch, starch, silica, a paraffinic hydrocarbon, an alkane, oat flour, clay, and petroleum. A fragrance and preservative portion 30 including fragrances and preservatives is added.

These and other objects, features, and advantages of the present invention will become more readily apparent from the attached drawings and the detailed description of the preferred embodiments, which follow.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A powder vehicle for the treatment of alopecia, the powder vehicle comprising:
minoxidil;
an alcohol;
arrowroot powder;
cornstarch;
starch;
fragrance;
silica;
a paraffinic hydrocarbon;
an alkane;
oat flour;
clay; and
petroleum;
wherein the paraffinic hydrocarbon is butane;
wherein the alkane is isobutene; and
wherein the powder vehicle is configured to assist in the growth of hair on the scalp and absorb excess grease and oil from the scalp.
